(19) United States
(12) Patent Application Publication
(10) Pub. No.: US 2009/0103745 A1
(43) Pub. Date: Apr. 23, 2009

(54) Headset with Active Noise Compensation

(76) Inventors: Axel Grell, Burgdorf (DE); Bjorn Wolter, Hannover (DE); Elmar Schulze, Lehrte (DE)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/162,019

(22) PCT Filed: Jan. 8, 2008
(86) PCT No.: PCT/EP08/50118

§ 371 Date: Nov. 21, 2008

(30) Foreign Application Priority Data
Jan. 8, 2007 (DE) 102007001980.9

(51) Int. Cl. G10K 11/16; A61F 11/06

(52) U.S. Cl. 381/71.6; 381/72

(57) ABSTRACT

There is provided an earphone comprising an electroacoustic reproduction transducer, a microphone (40) for recording interference sound and an active noise compensation unit for compensating for noise based on the interference sound recorded by the microphone (40). The earphone further has a housing (10) for accommodating the microphone (40) and the electroacoustic reproduction transducer. The housing (10) further has a first housing portion (50) which encloses at least the microphone (40) in such a way that a defined acoustic volume is obtained.

The present invention concerns an earphone or an ear protector.

Headphones or headsets with active noise compensation which is based for example on a feedback regulation technology have long been known.

DE 195 13 111 discloses a closed headset with a microphone and a reproduction transducer as well as a compensation circuit for sound compensation. In addition, provided in the headset is a compensating element which serves to cover over the sound entry opening of the microphone for reducing the effect of reflections at the ear. The compensating element has a plate with projections directed towards the reproduction transducer. The microphone is accommodated by a sleeve with a peripherally extending ridge.

DE 693 15 792 T2 discloses a telephone handset with a noise suppression function. The handset has at an earphone end an electroacoustic transducer, a microphone for recording interference sound, an active noise compensation unit and a housing. The microphone for recording interference sound is surrounded by a microphone housing and an earpiece.

DE 38 43 292 A1 discloses an earphone arrangement for headphones. The earphone arrangement has an electroacoustic transducer and a resonance wall.

As the human outer ear in each human being is of an individual configuration the reflections at the ear are different in each human being. Thus the compensating element can provide for defined sound reflection for the microphone.

In the case of headphones of that kind with active noise compensation or interference sound compensation the regulating loop for active noise compensation however can become unstable under certain conditions and rise. That can occur in particular when the ear or parts of the ear form a very small volume, in which case both the microphone and also the reproduction transducer are in that small volume.

Therefore an object of the present invention is to provide headphones or a headset or an ear protector which at least reduces instabilities in active noise compensation.

That object is attained by headphones as set forth in claim 1.

Thus there is provided an earphone comprising an electroacoustic reproduction transducer, a microphone for recording interference sound and an active noise compensation unit for compensating for noise based on the interference sound recorded by the microphone. The earphone further has a housing for accommodating the microphone and the electroacoustic reproduction transducer. The housing also has a first housing portion which encloses at least the microphone in such a way that a defined acoustic volume is obtained.

The invention is based on the concept of providing an acoustic volume in a housing of an earphone, in particular headphones or a headset, which cannot be altered by the user or by the geometry of the body of the user. Thus together with the regulating microphone, the sound reproduction transducer and the electronic system for active noise compensation it is possible to form a stable regulating system. The defined acoustic volume can be connected to the volume of the ear through defined holes. A material gauze can also be applied to the portion delimiting the acoustic volume.

Thus a defined acoustic volume can be formed around the regulating microphone in order to avoid possibly occurring instabilities and a feedback howl.

Further configurations of the invention are subject-matter of the appendant claims.

The advantages and embodiments by way of example of the invention are described in greater detail hereinafter with reference to the drawing.

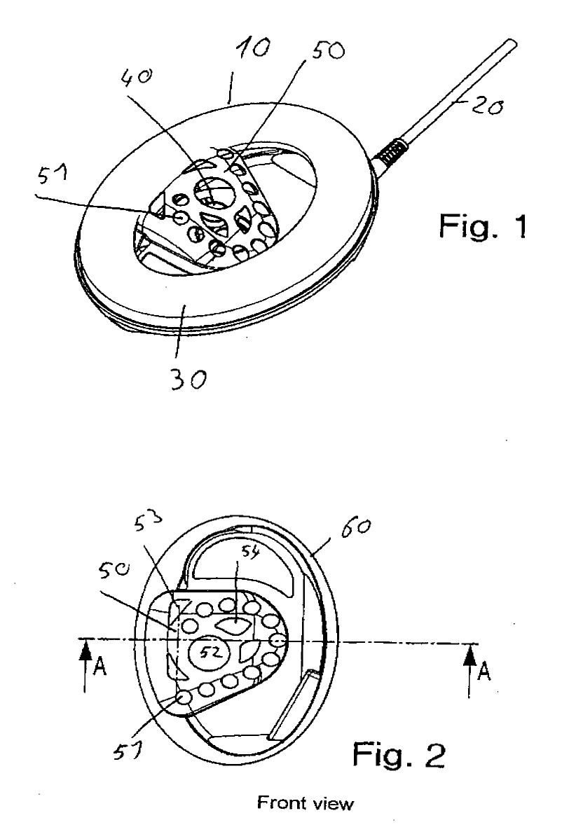

FIG. 1 shows an isometric view of a housing for a reproduction transducer of an earphone,

FIG. 2 shows a plan view of a housing of a reproduction transducer,

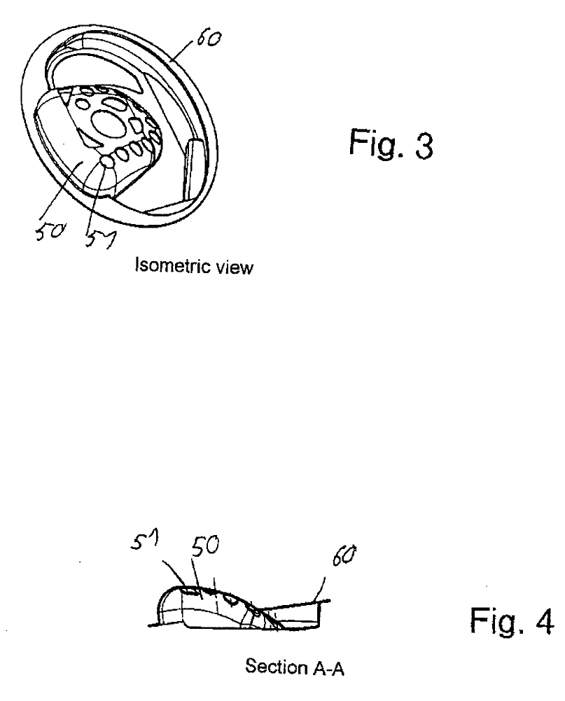

FIG. 3 shows an isometric view of the housing of FIG. 2, and

FIG. 4 shows a sectional view on section A-A of the housing of FIG. 2.

FIG. 1 shows an isometric view of a housing of a reproduction transducer of an earphone, in particular a headset or headphones. FIG. 1 thus shows a housing 10 of a reproduction transducer of a headset with a cable 20. An ear cushion 30 is fitted to the housing 10. The housing 10 further has a reproduction transducer and a microphone 40. The sound recorded by the microphone 40 is fed to an active noise compensation unit in order to subtract the recorded interference sound from the audio signal to be reproduced so that active noise compensation can be achieved. Also provided in the housing 10 is a first housing portion 50 which serves to form a defined acoustic volume around the microphone 40. The first housing portion 50 can have a large number of holes 51. The first housing portion 50 can be integrally connected to the housing 10.

FIG. 2 shows a plan view of a housing part 60 of the housing of FIG. 1. In this case the first housing portion 50 is fixed to or secured with the housing part 60. In this respect the first housing portion 50 can be formed or provided integrally with the housing part 60. As an alternative thereto the first housing portion 50 can be secured or glued to the housing part 60.

FIG. 3 shows an isometric view of the housing part 60 of FIG. 2.

FIG. 4 shows a sectional view along section line A-A of FIG. 2. Shown here are both the housing part 60 and also the first housing portion 50. In this case the first housing portion 50 has at least in part a predetermined curvature.

The provision of the first housing portion 50 which in relation to the regulating microphone 40 provides a defined acoustic volume means that it is possible to prevent instability in the regulating loop by virtue of a change in the acoustic volume in the region of the regulating microphone. The unwanted feedback howl can also be reduced in that way. The acoustic volume defined by the first housing portion 50 with the regulating microphone disposed therein and the sound reproduction transducer and the electronic system for active noise compensation thus represents a stable regulating system.

The first housing portion 50 preferably has at least one hole 51 which serves to communicate the acoustic volume defined by the first housing portion 50 with the volume of the ear. In addition thereto a material gauze can be provided in the region of the first housing portion 50.

Arranged in the first housing portion 50 are a number of round holes 51 as well as a larger round hole 52 in the central region of the first housing portion 50. Two substantially triangular holes 53 are provided in the connecting region between the housing part 60 and the first housing portion 50, a corner of the hole being of a rounded-off configuration. Two holes 54 are also provided in the central region of the first housing portion.

The first housing portion can serve both for accommodating the microphone and also for at least partially accommodating the electroacoustic reproduction transducer.

In accordance with a further embodiment there is provided a headset based on the above-described earphone. For that purpose it is only necessary to provide a further microphone for recording speech signals.

The above-described earphone can be for example in the form of headphones or an in-ear earphone or earbud.

1. An earphone comprising
an electroacoustic reproduction transducer,
a microphone (40) for recording interference sound,
an active noise compensation unit for compensating for noise based on the interference sound recorded by the microphone (40), and
a housing (10) for accommodating the microphone (40) and the electroacoustic reproduction transducer,
wherein the housing (10) has a first housing portion (50) which encloses at least the microphone (40) in such a way that a defined acoustic volume is obtained which is not variable by the user, wherein a material gauze is fitted over the first housing portion (50),
wherein the reproduction transducer is at least partially surrounded by the first housing portion (50).

2. An earphone according to claim 1 wherein the first housing portion (50) has at least one hole for communicating the acoustic volume define by the first housing portion (50) with an ear volume.

3. A headset comprising
a first microphone for recording speech signals,
an electroacoustic reproduction transducer,
a second microphone (40) for recording interference sound,
an active noise compensation unit for compensating for noise based on the interference sound recorded by the second microphone (40), and
a housing (10) for accommodating the second microphone (40) and the electroacoustic reproduction transducer,
wherein the housing (10) has a first housing portion (50) which encloses at least the second microphone (40) in such a way that a defined acoustic volume is obtained, which is not variable by the user, wherein a material gauze is fitted over the first housing portion (50),
wherein the reproduction transducer is at least partially surrounded by the first housing portion (50).

4. (canceled)

5. (canceled)
